(19) United States
(12) Patent Application Publication
(10) Pub. No.: US 2010/0129408 A1
(43) Pub. Date: May 27, 2010

(54) KLEBSIELLA PNEUMONIAE ATTENUATED VIRULENCE MUTANT AND METHOD OF PRODUCTION

(75) Inventors: Jin-Town Wang, Taipei City (TW); Pei-Fang Hsieh, Taipei City (TW); Tzu-Lung Lin, Taipei City (TW); Cha-Ze Lee, Taipei City (TW); Shih-Feng Tsai, Miaoli County (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL TAIWAN UNIVERSITY, Taipei City (TW)

(21) Appl. No.: 12/434,520

(22) Filed: May 1, 2009

(30) Foreign Application Priority Data
Nov. 26, 2008 (TW) 097145798

(51) Int. Cl. A61K 39/108; C12N 1/20; C12Q 1/68

(52) U.S. Cl. 424/259.1; 435/252.1; 435/6

(57) ABSTRACT

The present invention relates to a Klebsiella pneumoniae gene-deleted mutant and its producing methods. The main objective of this invention is to construct a Klebsiella pneumoniae tonB deletion mutant used as an immunogenically effective amount of a live attenuated vaccine against the community-acquired pyogenic liver abscess, where the tonB gene was deleted. The tonB deletion mutant maintained the structure of the capsular polysaccharide on cell membrane surface, and induced no disease on the mouse after injection. In addition, the tonB deletion mutants induced the immunity against pyogenic liver abscess caused by Klebsiella pneumoniae.

### BACKGROUND OF THE INVENTION

1. Field of the Invention

The present invention relates to a Klebsiella pneumoniae tonB deletion mutant and its producing methods. More particularly, the present tonB deletion mutant is used as an immunogenically effective amount of a live attenuated vaccine against the community-acquired pyogenic liver abscess.

2. Background of the Invention

Klebsiella pneumoniae belongs to the family of Enterobacteriaceae; which is Gram-negative, flagella-less and motionless bacterium with polysaccharide capsules. Strains of Klebsiella pneumoniae exist in the respiratory tract, digestive tract or urinary tract of healthy people. It's an opportunistic infectious pathogen. Infection results in pneumonia, urinary tract infections, or infections after surgical operation or intubation and the like. Klebsiella pneumoniae has become the predominant pathogen causing community-acquired pyogenic liver abscess (PLA) according to previous studies since 1986, which is an emerging endemic infectious disease particularly common in Asia and South Africa areas. Among the PLA patients, 10-13% combined with meningitis or endophthalmitis, and 40% to 78% of the patients had prior diabetes mellitus.

The cases of community-acquired PLA caused by Klebsiella pneumoniae have increased year by year from recent epidemiologic studies in Korea, Taiwan and USA. Characterization studies using Ribotyping and Pulsed-Field gel electrophoresis have excluded the homology of these Klebsiella pneumoniae strains. It's shown that PLA was not caused by one or a few specified epidemic strain but predominate a capsular subtype of K1. And magA (mucoviscosityassociated gene) is one of the genetic determinants of capsular serotype K1.

The known virulence factors of PLA-causing Klebsiella pneumoniae include not only K1 capsular associated antigens, magA, but also Allantoin metabolic gene, iron uptake gene kfu, and a regulatory gene for capsular exopolysaccharide synthesis, rmpA gene (regulator of the mucoid phenotype A). The general virulence factors include: (1) capsule, which prevents phagocytic destruction and complement serum lysis; and K1, K2 are the most virulent serotypes of the 77 recognized serotype strains; (2) lipopolysaccharide (endotoxin), elicits an immune reaction which is responsible for many of the harmful effects seen in septic shock, or provide serum resistance to avoid complement-mediated lysis; (3) adhesins, 5 types such as type 1, type 3, Klebsiella pneumoniae F-28 fimbriae, CF29K, and aggregative adhesion were identified, that are major factors in urinary tract and, respiratory tract infection since they facilitate the attachment of bacterium to the surface of host mucous; (4) siderophores, an iron chelating compound secreted by pathogenic bacteria to compete for iron with host iron-binding proteins since the concentration of free iron ions are very low in human bodies. Klebsiella pneumoniae acquires iron ion through synthesizing and secreting siderophores or hemophores to capture iron or heme, and using specialized outer membrane receptor to transport the irons. In addition, all siderophores, hemophores and outer membrane receptors shared the same characteristics: low similarity in N-terminal sequences, requiring a TonB complex which consists of 3 cytoplasmic membrane proteins: TonB, ExbB and ExbD. This TonB complex transduces energy to facilitate the iron internalization. The active transportation is coupled through gradients of concentration.

Klebsiella pneumoniae Studies toward community-acquired PLA or non-community PLA using transposon mutagenesis, full genome expression analysis and comparative genomic hybridization successfully identified virulence genes magA, allS and kfu. Further analysis by polymerase chain reaction (PCR) showed that these genes were mainly found in clinical isolates from patients of community-acquired PLA but not from non-community PLA. The induction of pathogenic genes of Klebsiella pneumoniae may be induced by environmental pressure during infection.

Recently, capsular serotype K1 was found to be the major virulence strain for Klebsiella pneumoniae causing PLA. And magA is one of the major components in capsular genetic determinants. Mutations in magA lost the exopolysaccharides and became avirulent. Therefore, the magA deletion mutant is not considered to be an ideal vaccine strain because of the lack of exopolysaccharides and immunogenicity.

Klebsiella pneumoniae vaccines using purified capsules of Klebsiella pneumoniae and other non-capsular compositions such as lipopolysaccharides, type 3 pilus, or even capsular gene deletion mutants were developed but with little success. Generally patients with PLA have been treated with catheter drainage and antibiotic therapy, but still showed a mortality of 10-40%. There is room for improvement since inactivated dead bacteria used in vaccine preparation limited the efficiency of antibody induction.

### SUMMARY OF THE INVENTION

The present invention provides a novel Klebsiella pneumoniae tonB deletion mutant and a producing method based on the research of previous studies to improve the efficiency of antibody induction.

The objective of the present invention is to provide a Klebsiella pneumoniae tonB deletion mutant used as an immunogenically effective amount of a live attenuated vaccine. The tonB deletion mutant was stored in Bioresource Collection and Research Center (Food Industry Research and Development Institute, Hsinchu, Taiwan) with an accession number of BCRC 910405. The live attenuated vaccine used for immunization against Klebsiella pneumoniae caused PLA has no pathogenicity to BALB/c mouse after intraperitoneal injection.

Another object of the present invention is to provide a method for producing the Klebsiella pneumoniae tonB deletion mutant. The tonB deletion mutant contains exopolysaccharides K1 and serum resistance, and grows in a condition containing iron ions. And the live attenuated vaccine further comprises a pharmaceutical carrier, diluent, or excipient.

The abovementioned method for producing a Klebsiella pneumoniae tonB deletion mutant comprises the steps of: (a) constructing a plasmid containing a tonB gene of Klebsiella pneumoniae and flanking regions of the tonB gene using a first primer set and a second primer set; (b) digesting the plasmid with a restriction enzyme to form a restriction fragment containing the flanking regions of tonB gene, and ligating the restriction fragment with a vector containing a temperature-sensitive region, an antibiotic selection marker and a negative selection marker; (c) transforming the vector into a Klebsiella pneumoniae strain; and (d) screening the tonB deletion mutant by an antibiotic and a negative selection drug, and obtaining the tonB deletion mutant containing the directly ligated flanking regions without tonB gene, and no selection markers.

The first primer set of step (a) is SEQ ID NO: 43 and SEQ ID NO: 44, and the second primer set is SEQ ID NO: 45 and SEQ ID NO: 46, the plasmid is a TA plasmid. The restriction enzyme of step (b) is NotI, and the vector is a pKO3 plasmid containing a temperature-sensitive origin, an antibiotic selection marker and a negative selection marker. The screened tonB deletion mutant was proved to be not pathogenicity to BALB/c mouse after activity assay but could be used for immunization against PLA—causing Klebsiella pneumoniae to induce anti-EPS IgG.

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### BRIEF DESCRIPTION OF THE DRAWINGS

FIG. 1. Diagram of microarray gene expression profile of cloned fragments in the present invention.

FIG. 2a. The genes of Yersinia HPI and iucABCDiutA distributed in Klebsiella pneumoniae genome.

FIG. 2b. The genes of iroA and hmuRSTUV distributed in Klebsiella pneumoniae genome.

FIG. 3a. Construction of the deletion mutants of [symbol] irp2 and [symbol] iuc.

FIG. 3b. Construction of the deletion mutants of [symbol] iroA and [symbol] kfu.

FIG. 4. The flow chart of the unmarked deletion method using a pKO3 plasmid.

FIG. 5. The growth curves of the NTUH-K2044 strain and other deletion mutants in iron-deficient conditions.

FIG. 6. The survival curves of BALB/c mice after infection of each deletion mutants.

FIG. 7. Results of injection with the NTUH-K2044 strain to the immunized BALB/c mice.

### DETAILED DESCRIPTION OF THE PREFERRED EMBODIMENT

The present invention used polysaccharide-protein conjugate vaccine which is the most effective vaccine.

The known virulence factors of PLA-causing Klebsiella pneumoniae include capsular associated antigens, metabolic gene, iron uptake gene, and a regulatory gene for capsular exopolysaccharide synthesis. Among them, the gene involved in iron transport system, such as tonB, is important since iron can be used in anti-oxidation of the hosts. Therefore, several Klebsiella pneumoniae deletion mutants are constructed in the present invention to against PLA causing Klebsiella pneumoniae. The non-pathogenic infection tonB deletion mutant was screened as the major active ingredient of vaccine candidates because it can induce the immunity to the same pathogens.

Single dose of tonB deletion mutant was found to be enough for the BALB/c mice to generate immunity from the screening studies on deletion mutants of PLA causing Kiebsiella pneumonia in the present invention. This showed that the tonB deletion mutant is able to stimulate BALB/c mice to generate protective memory immune responses. The tonB deletion mutant would not be cleaned immediately by the immune responses of the hosts since it contains complete K1 capsular exopolysaccharides, serum resistance and also induces anti-EPS IgG production in comparison to magA mutants.

The original Klebsiella pneumoniae strain of the tonB mutant was screened and isolated from the blood of the PLA patient in National Taiwan University Hospital (NTUH) with an accession number of NTUH-K2044. The whole genome size is 5.2×10⁶ bases, including about 5093 open reading frames (ORFs), a GC ratio of 57.68% and a plasmid pK2044 (large plasmid) of 2.24×10⁵ bases, 299 ORFs, a GC ratio of 50.17%.

All the containers for NTUH-K2044 RNA extraction were washed by a solution of 0.1% DEPC to prevent the degradation of RNase in the air.

Overnight culture of NTUH-K2044 Klebsiella pneumoniae were cultured in 1 ml of LB media and 1 ml of diluted healthy human blood at 4% to 8%, or in 2 ml of LB for the control group. The cells were grown at 37° C. for 3-4 h to log phase, then centrifuged at 14000 g, 4° C. for 2 h. The supernatant was removed and the cell pellet was washed twice with 0.1% DEPC ddH₂O. The cells were centrifuged again for 2 h then the supernatant was removed. Each ml of the cell pellet solution was resuspended into 0.25 ml of lysis buffer and then boiled at 100° C. for 5 min till solution was clear. The RNA of the strain was isolated with RNeasy Mini Kit (Qiagen, cat. No. 74106) and residual DNA was removed with RNase-Free DNase Set (cat. No. 79254) after cool down. RNA concentration and purity was determined using agarose electrophoresis and spectrophotometrically at OD_{260/280}.

Microarray hybridization was used to analyze the expression level of extracted RNA from NTUH-K2044. To 5 μl of the RNA, 7.5 μl of random hexamer primer and 14.75 μl of DEPC-dH₂O were added and heated at 70° C. for 3 min, followed by addition of 12.5 μl of 5× First RT buffer, 0.1 M DTT, and 2.5 μl each of dATP, dCTP, dGTP, and dTTP (in the concentration of 25 μl), 6 μl of biotin-16-dUTP (25 mM), 1.5 μl of RNaseOut™ Ribonuclease and 5 μl of Superscript II reverse transcriptase. The reverse transcription-PCR (RT-PCR) was carried out at 25° C. for 10 min, 42° C. for 90 min and 94° C. for 5 min. The biotin-16-dUTP could then be incorporated into the first strand of cDNA. This biotin-16-dUTP cDNA can be used as labeled probe after incubated with 6.9 μl NaOH (3M) at 50° C. for 30 min to remove the residual RNA and neutralized with 6.9 μl acetic acid (3M) at 50° C. for 30 min. The labeled probe can be precipitated with ethanol.

The microarray filter was incubated with 2 ml of hybridization buffer at 63° C. for 1.5-2 h. One ml of the hybridization buffer containing the labeled probe was added onto the surface of filter and incubated at 63° C. for 16-18 h. The filter was washed with 2×SSC and 0.1% SDS twice at room temperature (evenly shaking) for 5 min, with a final wash of 0.1×SSC and 0.1% SDS three times at 63° C. for 15 min.

Color development was performed after the microarray filter was treated in the following order: incubating with 2 ml of blocking buffer with shaking at room temperature for 1 h, incubating with 2 ml of reaction buffer at room temperature for 1 h, washing with PBST buffer for 4 times at room temperature for 5 min each, reacting with 1.5 ml of NBT/BCIP buffer at room temperature for 20-40 min, and stopping the reaction with water. The color developed in the filter was scanned in a scanner and stored in the computer followed by quantification of the images with NIH ImageJ software. The 23SrRNA was served as the internal control.

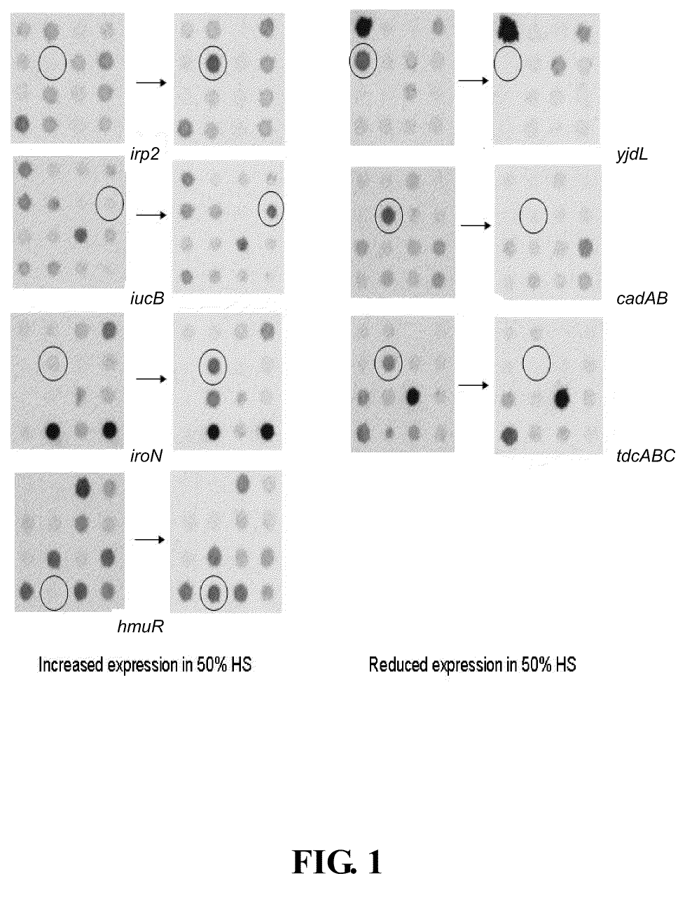

Referring to FIG. 1, the microarray gene expression profile of 14 cloned sequences, including Yersinia HPI iucABCDiutA, iroA and hmuRSTUV, showed significant increase of RNA expression levels in human serum-induction group but not in control group. Yersinia HPI, iucABCDiutA and iroA belong to the siderophore-dependent iron acquisition systems, while hmuRSTUV is an ATP-binding cassette (ABC) hemoprotein transport system. In contrast, 5 clones revealed a decrease in RNA expression level, including yjdL, cadABC and tdcABCDE gene loci.

### Example 1

### Method for Establishing Klebsiella Pneumoniae Gene Deletion Mutants

All the cloned fragments with higher gene expression levels than the control group were selected as candidates of deletion mutants. The method for producing a Klebsiella pneumoniae tonB deletion mutant comprises the steps of: (a) constructing a plasmid containing a tonB gene of Klebsiella pneumoniae and flanking regions of the tonB gene using a first primer set and a second primer set; (b) digesting the plasmid with a restriction enzyme to form a restriction fragment containing the flanking regions of tonB gene, and ligating the restriction fragment with a vector containing a temperature-sensitive region, an antibiotic selection marker and a negative selection marker; (c) transforming the vector from step (b) into a Klebsiella pneumoniae strain; and (d) screening the tonB deletion mutant by an antibiotic and a negative selection drug, and obtaining the tonB deletion mutant containing the directly ligated flanking regions without tonB gene, and no selection markers. The abovementioned primer sets were listed in Table 1:

| | | | |
|---|---|---|---|
| primers | sequence | usage | SEQ ID NO |

| | | | |
|---|---|---|---|
| 1040-F | GGTGCTCTTTACATCATTGC | prevalence analysis | SEQ ID NO:1 |
| 936-R | GCAATGGCCATTTGCGTTAG | magA | SEQ ID NO:2 |
| 10E4-2-5F | AGTCGGCCTGGGGTTTAAGG | prevalence analysis allS | SEQ ID NO:3 |
| 10E4-2- | CAGTCAACGTGGCGATTCGC | | |
| 475R | | | |
| kfu-F1179 | GAAGTGACGCTGTTTCTGGC | prevalence analysis | SEQ ID NO:5 |
| kfuC-R649 | TTTCGTGTGTGGCCAGTGACTC | kfu/PTS | SEQ ID NO:6 |
| ybtU-F | TTGTGCGCAACACATTACGC | prevalence analysis | SEQ ID NO:7 |
| ubtU-R | TCACAGCGCCTCCTTATCAT | Yersinia HPI | SEQ ID NO:8 |
| ybtA-F | ATGACGGAGTCACCGCAAAC | | SEQ ID NO:9 |
| ybtA-R | TTACATCACGCGTTTAAAGG | | SEQ ID NO:10 |
| iucB-F | ATGTCTAAGGCAAACATCGT | iucABCDiutA prevalence | SEQ ID NO:11 |
| iucB-R | TTACAGACCGACCTCCGTGA | analysis & iucB qPCR | SEQ ID NO;12 |
| iroN-F | GTCCGGCGGTAACTTCAGCC | iroA(iroNDCB) | SEQ ID NO:13 |
| iroN-P-R | TCAGAATGAAACTACCGCCC | prevalence analysis & | SEQ ID NO:14 |
| | | iroN qPCR | |
| iroNB-F | GGCTACTGATACTTGACTATTC | iroA(iroBCDN) | SEQ ID NO:15 |
| iroNB-R | CAGGATACAATAGCCCATAG | prevalence analysis | SEQ ID NO:16 |
| hmuR-F | GTGGCGACTATGTACAAATC | hmuRSTUV prevalence | SEQ ID NO:17 |
| hmuR-R | GCTGTTGTTTTCAGTTTCCT | analysis & human qPCR | SEQ ID NO:18 |
| KP-23-F | GGTTAAGCGACTAAGCGTACACGGT | 23S rRNA qPCR | SEQ ID NO:19 |
| KP-23-R | ACGAGGCGCTACCTAAATAGCTTTC | | SEQ ID NO:20 |
| irp2-2200F | GCATGACAGGGTGCTGGCCC | irp2 qPCR | SEQ ID NO:21 |
| irp2-2899R | CTCCGACTTTGACCTGCTTGTC | | SEQ ID NO:22 |
| ybtA-F | ATGACGGAGTCACCGCAAAC | irp2 deletion construct | SEQ ID NO:23 |
| irp1-R | CGGTATAGCCGACCTTTCTG | | SEQ ID NO:24 |
| ybtA-R | TTACATCACGCGTTTAAAGG | | SEQ ID NO:25 |
| irp1-F | ATGGATAACTTGCGCTTCTC | | SEQ ID NO:26 |
| Iuc-FR | GAGCCGCCCCAAACGACAGC | iucABCDiutA deletion | SEQ ID NO:27 |
| Iuc-RR | GGCTTTTCTGATACCAATCT | construct | SEQ ID NO:28 |
| iuc-Fout | AAGCACAATCAATATATGG | | SEQ ID NO:29 |
| iuc-Rout | CCGGTATTCCTTTACAACAA | | SEQ ID NO:30 |
| iro-PF | TCCTGTTGGCCAGCGTCTAT | iroA(iroNDCB) deletion | SEQ ID NO:31 |
| iro-PR | CTCCTTCAGCCCGAACAAAC | construct | SEQ ID NO:32 |
| iro-P-Fout | TCGTAATTATTAGGACTAAG | | SEQ ID NO:33 |
| iro-P-Rout | GCTCCTGTATACTATGGCAG | | SEQ ID NO:34 |
| iroN-R | TCAGAATGATGCGGTGACAC | iroA(iroBCDN) deletion | SEQ ID NO:35 |
| iroD-R | TCAACCTTTTAGTAAACC | construct | SEQ ID NO:36 |
| iroN-F | GTCCGGCGGTAACTTCAGCC | | SEQ ID NO:37 |
| iroD-F | ATGCTGAACATGCAACAAC | | SEQ ID NO:38 |
| kfu-FR | GCAGCAGATGAATATTCTGG | kfuABC deletion | SEQ ID NO:39 |
| kfu-RR | TTCCGACGCCAATGCTGATC | construct | SEQ ID NO:40 |
| kfu-Fout | TCTGGGTGCAGAACCAAATG | | SEQ ID NO:41 |
| kfu-Rout | ATGGAGTGGTAGTACGTTGG | | SEQ ID NO:42 |
| tonB-FR | AGCAACTTAACGCTGGCAGC | tonB deletion construct | SEQ ID NO:43 |
| tonB-RR | TGAGCTGGGTACCAACACC | | SEQ ID NO:44 |
| tonB-Fout | GCAATCATATTCAATAAGG | | SEQ ID NO:45 |
| tonB-Rout | TAAAACGCTGCGGCGGACCG | | SEQ ID NO:46 |
| iro-pP | GAAAATCCCTCTTTTAACGC | salmochelin receptor | SEQ ID NO:47 |
| iroN-P-R | TCAGAATGAAACTACCGCCC | IroN from | SEQ ID NO:48 |
| | | iroA(iroNDCB) | |
| iro-pC | TCGATCCGGTTGTTTGCAGG | salmochelin receptor | SEQ ID NO:49 |
| iroN-R | TCAGAATGATGCGGTGACAC | IroN from | SEQ ID NO:50 |
| | | iroA(iroBCDN) | |
| irp2-p | CCCCTTCGACCTTTAAACGC | irp2 complementation | SEQ ID NO:51 |
| irp2-R | CTATATCCGCCGCTGACGAC | | SEQ ID NO:52 |
| pIuc-F | CCAGTACAGGGATCGCGACC | iucABCDiutA | SEQ ID NO:53 |
| iutA-R | TCAGAACAGCACAGAGTAGTTCAG | complementation | SEQ ID NO:54 |
| tonB-F | CGTAAAGCACGGCAAAGCTC | tonB complementation | SEQ ID NO:55 |
| tonB-R | TCAGTTAATCTCGACGCCG | | SEQ ID NO:56 |

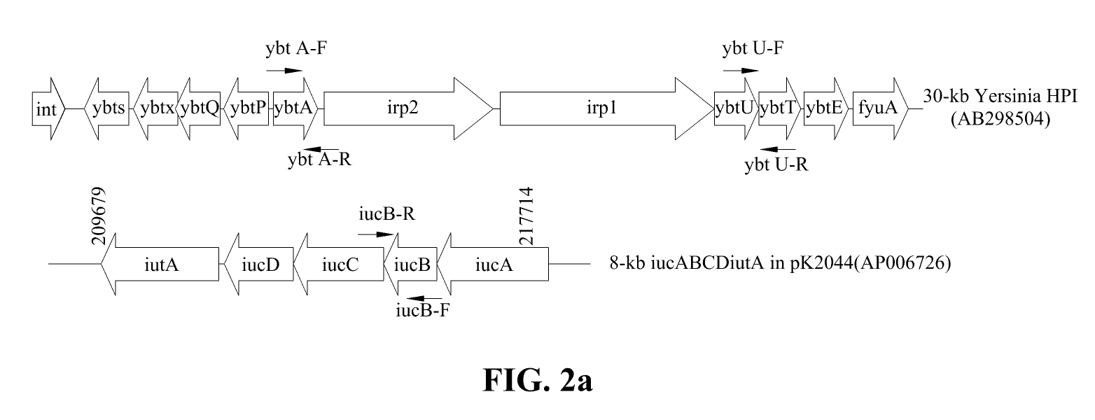

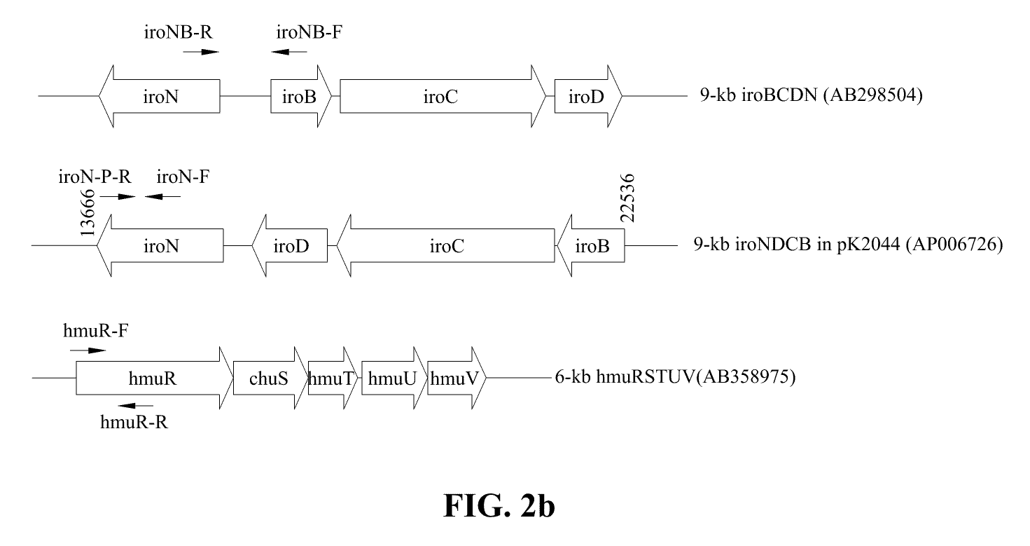

Primer sets listed in the above table and PCR performed in step (a) were used as reference for gene deletion to detect the distribution of gene fragments such as Yersinia HPI, iucABCDiutA, iroA and hmuRSTUV in community acquired PLA or non-community acquired PLA. The cycling program consisted of one denaturation step of 3 min at 96° C. and 30 cycles of 30 s at 96° C., 15 s at 52° C., and 1 min at 74° C., followed by 10 min at 72° C. Referring to FIGS. 2a and 2b, respectively the gene distribution diagram of Yersinia HPI and iucABCDiutA, iroA and hmuRSTUV in Klebsiella pneumoniae genome was shown. The distribution of Yersinia HPI iucABCDiutA, iroA and hmuRSTUV was shown after PCR. These 4 genes were shown to be more common (associated with) in community acquired PLA than in non-community acquired PLA. Therefore, the deletion mutants of Yersinia HPI, iucABCDiutA, iroA and hmuRSTUV are candidates of the live attenuated vaccine against community acquired PLA.

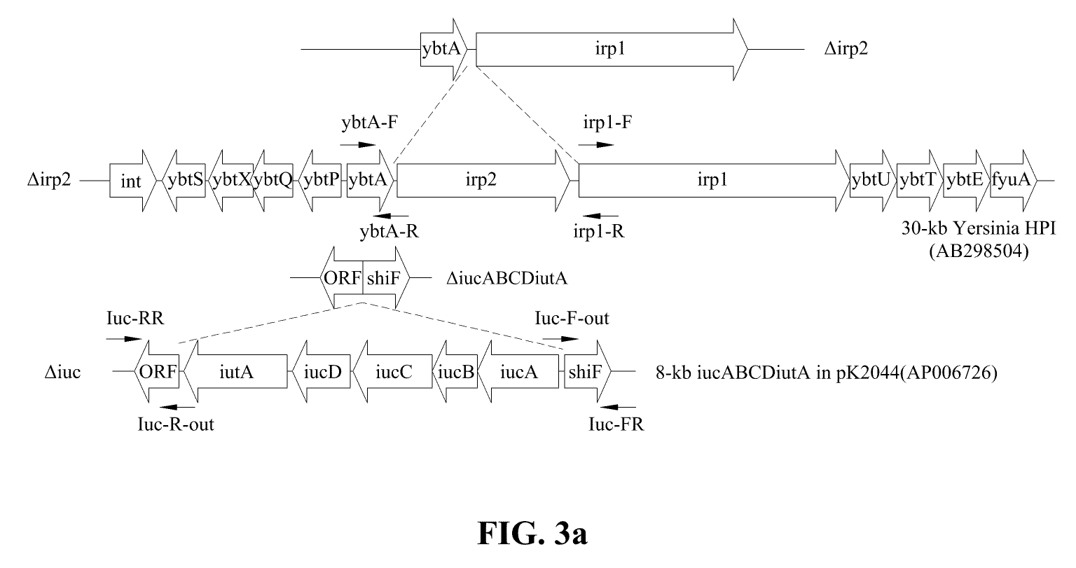

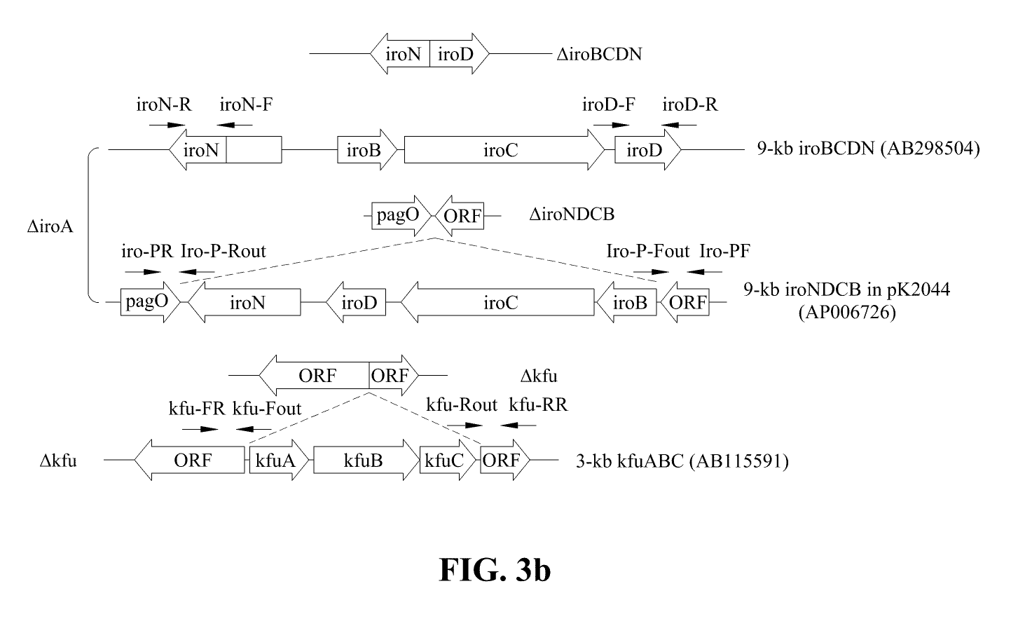

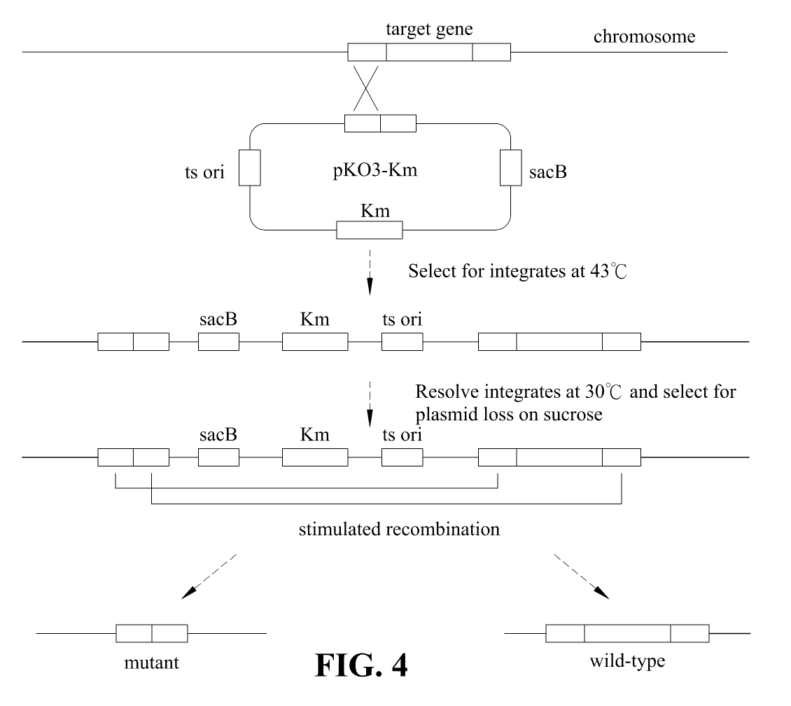

Referring to FIGS. 3a and 3b, respectively the deletion mutants of [symbol] irp2 and [symbol] iuc, [symbol] iroA and [symbol] kfu were constructed. Unmarked deletion method is one of the several methods used to construct the deletion mutants in the invention. The targeted deletion genes such as [symbol] irp2, [symbol] iuc, [symbol] iroA and [symbol] kfu in dotted lines were the few examples shown in FIG. 3. Other single or multiple gene deletion such as [symbol] tonB, [symbol] irp2 iuc iroA and [symbol] irp2 iuc iroA kfu can further be included. FIG. 4 shows the unmarked deletion method. The flanking regions of the target gene were ligated into the plasmid pKO3, a gene replacement vector that contains a temperature-sensitive origin of replication and markers for positive and negative selection for chromosomal integration and excision. The recombinant pKO3 plasmid was transformed into Klebsiella pneumoniae using electroporation and cultured at 43° C. in LB plates containing kanamycin. This plasmid would integrate into the chromosome of NTUH-K2044 through homologous recombination. PCR was carried out to screen the pKO3 integrated clones. These clones were cultured at 30° C. in LB plates containing 5% sucrose. The plasmid pKO3 can be generated again through homologous recombination to form gene deletion mutants of NTUH-K2044. PCR was carried out to screen the mutants.

The [symbol] tonB deletion mutant was taken as an example. Primers tonB-FR (SEQ ID NO: 43) and tonB-RR (SEQ ID NO: 44) were used to synthesize a complete tonB gene through PCR, including 1 kb of the upstream and downstream flanking regions in step (a). This gene fragment was ligated into a plasmid such as TA. A reverse PCR was performed using the resulting plasmid as a template, plus the primers tonB-Fout (SEQ ID NO: 45) and tonB-Rout (SEQ ID NO: 46, LA-Taq polymerase to yield products. The A-tail of the 3′-end was removed from the products using T4 DNA polymerase (NEB) and phosphate was attached to the 5′-end of the products using polynucleotide kinase (NEB) for self-ligation.

The restriction enzymes used to digest the tonB gene and the flanking regions from the TA cloning vector in Step (b) including but not limited to NotI, the vector containing temperature-sensitive region, an antibiotic selection marker and a negative selection marker including but not limited to pKO3, the antibiotic selection marker including but not limited to kanamycin, the negative selection marker including but not limited to sacB (encoding levansucrase).

Electroporation can be used in step (c) to transform the flanking regions of tonB gene into the NTUH-K2044 strain.

A tonB deletion mutant containing the directly ligated flanking regions without tonB gene was screened by the antibiotic and the negative selection marker in step (d), which contained no selection marker in the genome. The sequence of mutant was confirmed with PCR and sequence analysis. The negative selection marker was sucrose in step (d).

The functional analysis was carried out with trans-complementation, cross-feeding assay, growth study, String test, Serum resistance assays, serotyping, animal study, Enzyme-Linked Immunosorbent Assay (ELISA) and immunoblotting.

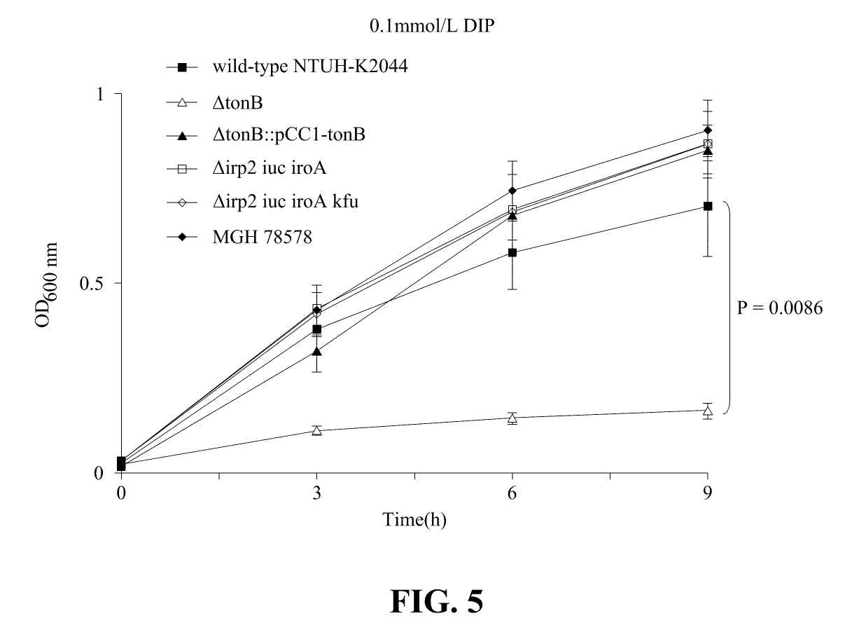

The growth curves of the NTUH-K2044 strain and each deletion mutants (irp2, iuc, or iroA single mutants, irp2 iuc iroA triple mutant and irp2 iuc iroA kfu quadruple mutant) were shown in FIG. 5. All mutant strains as well as the wild type parental strain showed no difference in both iron-replete and iron-deficient conditions in vitro. Wild-type NTUH-K2044 secreted yersiniabactin and aerobactin into the culture supernatants but the deletion mutants irp2 or iuc single mutants, triple mutant and quadruple mutant produced neither yersiniabactin nor aerobactin as determined by cross-feeding assays. However, plasmid CopyControl pCC1 containing corresponding genes used in trans-complementation restored the ability to produce yersiniabactin or aerobactin of these mutants as shown in Table 2.

TABLE 2

| | Genotype or | Synthesis | Synthesis of | | Serum |
|---|---|---|---|---|---|
| Strain | phenotype* | of Ybt^{†} | Aerobactin^{†} | K1 CPS Ag | resistance^{‡} |
| NTUH-K2044 | Wild-type/m⁺ | + | + | + | R |
| Δirp2 | irp2/m⁺ | − | ND | ND | ND |
| Δiuc | iuc/m⁺ | ND | − | ND | ND |
| ΔiroA | iroA/m⁺ | ND | ND | ND | ND |
| Δkfu | kfu/m⁺ | ND | ND | ND | ND |
| Δirp2 iuc iroA | irp2 iuc iroA/m⁺ | − | − | + | ND |
| Δirp2 iuc iroA | irp2 iuc iroA kfu/ | − | − | + | R |
| kfu | m⁺ | | | | |
| Δirp2::pCC1-irp2 | Irp2/m⁺ | + | ND | ND | ND |
| Δiuc::pCC1-iuc | Iuc/m⁺ | ND | + | ND | ND |
| ΔtonB | tonB/m⁺ | ND | ND | + | R |
| ΔtonB::pCC1- | TonB/m⁺ | ND | ND | + | ND |
| tonB | | | | | |

*m⁺, mucoviscosity;
+, synthesizing ability ≧5 mm;
ND, not detected;
^{‡}R, resistance.

The ΔtonB deletion mutant was shown to remain hyperviscous, K1 capsular exopolysaccharides, and serum resistance according to Table 2. Animal studies were carried out to test the possibility for mutants as vaccine candidates. Five-week-old female BALB/c mice were obtained from National Laboratory Animal Center. The BALB/c mice were infected intraperitoneally (IP) or intragastrically (IG) with Klebsiella pneumoniae NTIH-K2044 mutants including [symbol] irp2 iuc iroA triple mutant, [symbol] irp2 iuc iroA kfu quadruple mutant, [symbol] tonB mutant, [symbol] magA mutant (not able to synthesize K1 capsular exopolysaccharide) at the dose of 1×10³ CFU to [symbol] irp2 iuc iroA triple mutant, [symbol] irp2 iuc iroA kfu quadruple mutant, and 1×10⁴ CFU or 1×10⁵ CFU to [symbol] tonB mutant, and 1×10⁶ CFU to [symbol] magA mutant; saline was used in the control group. After 4 weeks, immunized and non-immunized control BALB/c mice were challenged with 1×10³ CFU of NTUH-K2044 (greater than the wild-type LD₅₀ value). The challenged mice were monitored for another 4 weeks. The pathogenicity was analyzed with Kaplan-Meier analysis (SPSS 12^{th} ed.) and a log-rank test; P<0.05 was considered to be statistically significant.

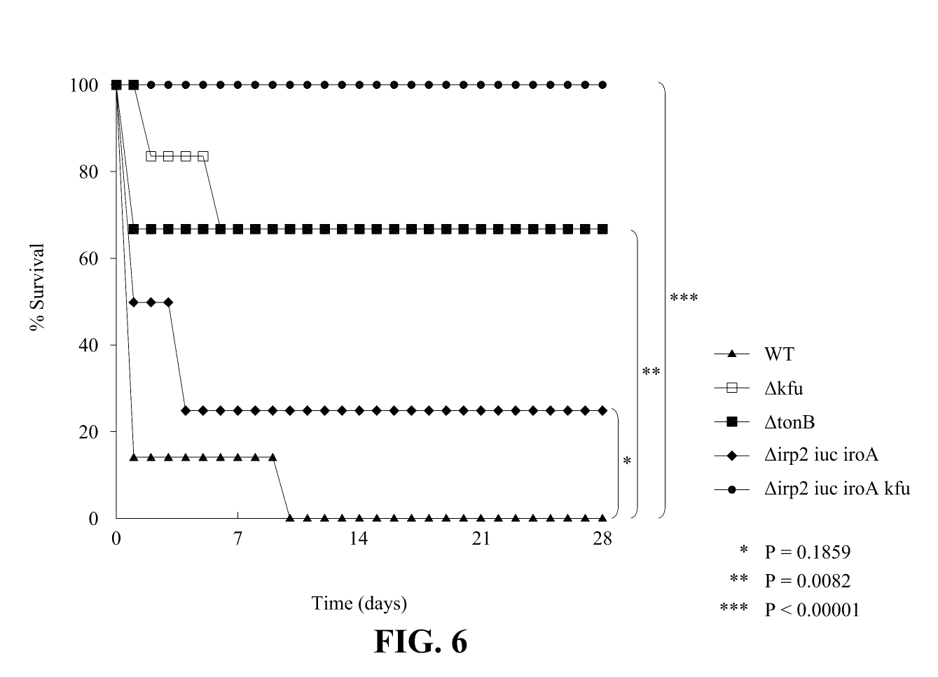

Single mutants including [symbol] irp2, [symbol] iuc, [symbol] iroA, and [symbol] kfu were shown to have the same pathogenicity with the wild type NTIH-K2044 (LD₅₀<1×10² CFU) after IP infection. However, the mice survived after injection of the dose of 1×10³ CFU to [symbol] irp2 iuc iroA triple mutant or [symbol] irp2 iuc iroA kfu quadruple mutant (LD₅₀ of 1.3×10⁴ CFU for irp2 iuc iroA triple mutant and 5.5×10⁴ CFU for irp2 iuc iroA kfu quadruple mutant). In addition, there was no significant difference for the pathogenicity between the [symbol] irp2 iuc iroA triple mutant and the wild type strain after IG feeding (LD₅₀ of 5.6×10⁵ CFU for irp2 iuc iroA triple mutant and 1×10⁵ CFU for the wild-type strain). Referring to FIG. 6, the pathogenicity of the [symbol] irp2 iuc iroA kfu quadruple mutant was even lower than the wild type strain or the [symbol] kfu mutant after IG feeding (LD₅₀ of >1×10⁷ CFU for irp2 iuc iroA kfu quadruple mutant and LD₅₀ of 6.3×10⁶ CFU for [symbol] kfu mutant). [symbol] tonB mutant was found to have no pathogenicity in mice after 28 days observation and the survival rate was 100%.

Analysis of the genomic sequence of Klebsiella pneumoniae NTUH-K2044 identified ten putative iron transport systems, and 7 of them were TonB dependent, such as Yersinia HPI, iucABCDiutA, and iroA. The tonB deletion mutant was unable to grow in iron-deficient media, whereas complementation of the tonB deletion mutants restored growth in iron-restricted media. The tonB deletion mutant was therefore confirmed to be defective in iron uptake. In comparison with the parental NTUH-K2044 strain, the tonB deletion mutant formed smaller colonies on either blood agar or LB media. IP inoculation of BALB/c mice with the tonB deletion mutant resulted in a less efficient spread in the body than IP injection of wild type strain at the same dose. A string test was examined in these deletion mutants and revealed that the tonB deletion mutant remained hyperviscous. There was no significant difference in CPS antigen serotyping using double immunodiffusion and serum sensitivity assays between the tonB mutant and the wild type NTUH-K2044 strain. Therefore, the immunogenicity was regarded the same for both strains.

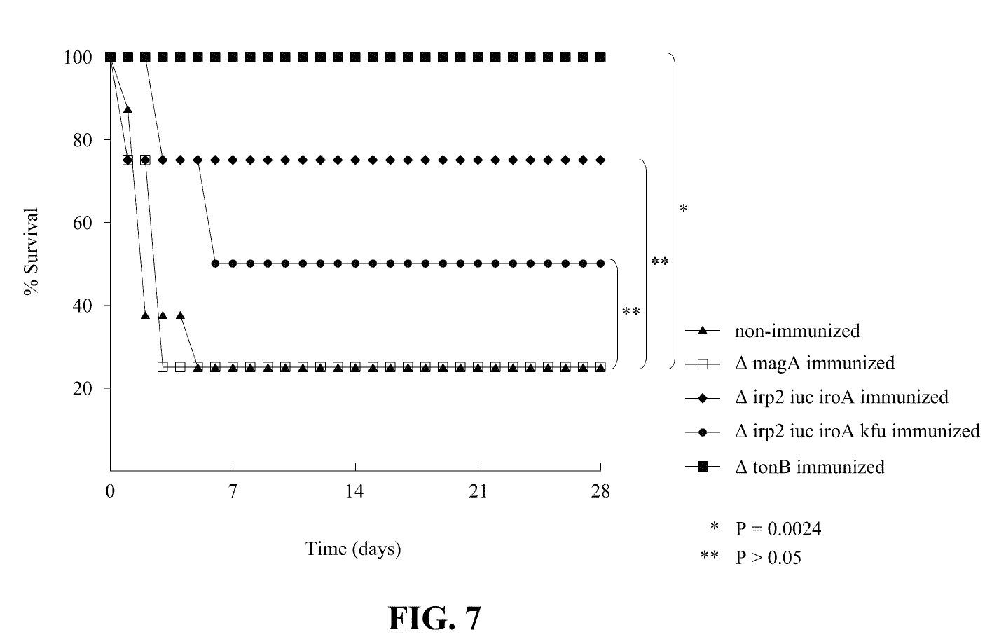

Referring to FIG. 7, the results of injection of NTUH-K2044 strain to the immunized BALB/c mice were shown. The protective efficacy of the live vaccines using mutants of magA, [symbol] irp2 iuc iroA, [symbol] irp2 iuc iroA kfu, and [symbol] tonB were compared with non-immunized BALB/c mice. These attenuated strains of each mutant were injected into BALB/c mice respectively to get immunity, followed by challenging with the lethal dose of NTUH-K2044. Four weeks later, all tonB deletion mutant-immunized BALB/c mice survived without any symptoms of disease, whereas 75% of the non-immunized control BALD/c mice or magA mutant-immunized BALB/c mice died within 5 days of infection. The survival rates of [symbol] irp2 iuc iroA and [symbol] irp2 iuc iroA kfu-mutant immunized BALB/c mice were lower than that of tonB mutant-immunized BALB/c mice. ELISA analysis showed that BALB/c mice immunized with the tonB mutant had EPS serum immunoglobulin G (anti-EPS IgG) responses against NTUH-K2044 strain, while control mice or magA mutant-immunized mice did not.

From the description and results of the abovementioned animal studies, the tonB deletion mutant was shown to be a potential live vaccine since the strain could survive in human blood and has the ability to synthesize K1 capsular exopolysaccharides. Therefore the tonB deletion strain can be prepared as live vaccine in large scale, resulting in induction of anti-EPS antibody in human and immediate protect against the Klebsiella pneumoniae—causing community acquired PLA. The tonB deletion mutant was stored in Bioresource Collection and Research Center (Food industry Research and Development Institute, Hsinchu, Taiwan) on Oct. 30, 2008 with an accession number of BCRC910405.

What is claimed is:

1. A Klebsiella pneumoniae tonB deletion mutant (BCRC No. 910405) used as an immunogenically effective amount of a live attenuated vaccine.

2. The tonB deletion mutant as claimed in claim 1, wherein the Klebsiella pneumoniae cause community-acquired pyogenic liver abscess (PLA).

3. The tonB deletion mutant as claimed in claim 1, wherein a serotype of the Klebsiella pneumoniae tonB deletion mutant is a K1 serotype.

4. The tonB deletion mutant as claimed in claim 1, wherein the tonB deletion mutant has no pathogenicity.

5. The tonB deletion mutant as claimed in claim 1, wherein the live attenuated vaccine further comprises a pharmaceutical carrier, diluent, or excipient.

6. The tonB deletion mutant as claimed in claim 1, wherein an anti-EPS IgG antibody is induced by the tonB deletion mutant.

7. The tonB deletion mutant as claimed in claim 1, wherein the tonB deletion mutant survives in a condition containing iron ions.

8. A method for producing a Klebsiella pneumoniae tonB deletion mutant (BCRC No. 910405), comprising:
(a) constructing a plasmid containing a tonB gene of Klebsiella pneumoniae and flanking regions of the tonB gene using a first primer set and a second primer set;
(b) digesting the plasmid with a restriction enzyme to form a restriction fragment containing the flanking regions of tonB gene, and ligating the restriction fragment with a vector containing a temperature-sensitive region, an antibiotic selection marker and a negative selection marker;
(c) transforming the vector into a Klebsiella pneumoniae strain; and
(d) screening the tonB deletion mutant by an antibiotic and a negative selection drug, and obtaining the tonB deletion mutant containing the directly ligated flanking regions without tonB gene, and no selection markers.

9. The method as claimed in claim 8, wherein the Klebsiella pneumoniae cause community-acquired pyogenic liver abscess (PLA).

10. The method as claimed in claim 8, wherein the first primer set is SEQ ID NO: 43 and SEQ ID NO: 44.

11. The method as claimed in claim 8, wherein the second primer set is SEQ ID NO: 45 and SEQ ID NO: 46.

12. The method as claimed in claim 8, wherein the plasmid of step (a) is a TA plasmid.

13. The method as claimed in claim 8, wherein the restriction enzyme of step (b) is NotI.

14. The method as claimed in claim 8, wherein the vector of step (b) is a pKO3 plasmid.

15. The method as claimed in claim 8, wherein the temperature-sensitive region of step (b) is a temperature-sensitive replication origin of Psc101.

16. The method as claimed in claim 8, wherein the antibiotic selection marker of step
(b) is an kanamycin resistance gene.

17. The method as claimed in claim 8, wherein the negative selection marker of step
(b) is a sacB gene.

18. The method as claimed in claim 8, wherein the negative selection drug of step (d) is sucrose.


SEQUENCE LISTING (56 sequences)

SEQ ID NO: 1 - DNA, 20 bp, Artificial Sequence
  prevalence analysis magA
ggtgctctttacatcattgc

SEQ ID NO: 2 - DNA, 20 bp, Artificial Sequence
  prevalence analysis magA
gcaatggccatttgcgttag

SEQ ID NO: 3 - DNA, 20 bp, Artificial Sequence
  prevalence analysis allS
agtcggcctggggtttaagg

SEQ ID NO: 4 - DNA, 20 bp, Artificial Sequence
  prevalence analysis allS
cagtcaacgtggcgattcgc

SEQ ID NO: 5 - DNA, 20 bp, Artificial Sequence
  prevalence analysis kfu/PTS
gaagtgacgctgtttctggc

SEQ ID NO: 6 - DNA, 22 bp, Artificial Sequence
  prevalence analysis kfu/PTS
tttcgtgtgtggccagtgactc

SEQ ID NO: 7 - DNA, 20 bp, Artificial Sequence
  prevalence analysis Yersinia HPI
ttgtgcgcaacacattacgc

SEQ ID NO: 8 - DNA, 20 bp, Artificial Sequence
  prevalence analysis Yersinia HPI
tcacagcgcctccttatcat

SEQ ID NO: 9 - DNA, 20 bp, Artificial Sequence
  prevalence analysis Yersinia HPI
atgacggagtcaccgcaaac

SEQ ID NO: 10 - DNA, 20 bp, Artificial Sequence
  prevalence analysis Yersinia HPI
ttacatcacgcgtttaaagg

SEQ ID NO: 11 - DNA, 20 bp, Artificial Sequence
  prevalence analysis iucABCDiutA & iucB qPCR
atgtctaaggcaaacatcgt

SEQ ID NO: 12 - DNA, 20 bp, Artificial Sequence
  prevalence analysis iucABCDiutA & iucB qPCR
ttacagaccgacctccgtga

SEQ ID NO: 13 - DNA, 20 bp, Artificial Sequence
  prevalence analysis iroA(iroNDCB) & iroN qPCR
gtccggcggtaacttcagcc

SEQ ID NO: 14 - DNA, 20 bp, Artificial Sequence
  prevalence analysis iroA(iroNDCB) & iroN qPCR
tcagaatgaaactaccgccc

SEQ ID NO: 15 - DNA, 22 bp, Artificial Sequence
  prevalence analysis iroA(iroBCDN)
ggctactgatacttgactattc

SEQ ID NO: 16 - DNA, 20 bp, Artificial Sequence
  prevalence analysis iroA(iroBCDN)
caggatacaatagcccatag

SEQ ID NO: 17 - DNA, 20 bp, Artificial Sequence
  prevalence analysis hmuRSTUV & hmuR qPCR
gtggcgactatgtacaaatc

SEQ ID NO: 18 - DNA, 20 bp, Artificial Sequence
  prevalence analysis hmuRSTUV & hmuR qPCR
gctgttgttttcagtttcct

SEQ ID NO: 19 - DNA, 25 bp, Artificial Sequence
  23S rRNA qPCR
ggttaagcgactaagcgtacacggt

SEQ ID NO: 20 - DNA, 25 bp, Artificial Sequence
  23S rRNA qPCR
acgaggcgctacctaaatagctttc

SEQ ID NO: 21 - DNA, 20 bp, Artificial Sequence
  irp2 qPCR
gcatgacagggtgctggccc

SEQ ID NO: 22 - DNA, 22 bp, Artificial Sequence
  irp2 qPCR
ctccgactttgacctgcttgtc

SEQ ID NO: 23 - DNA, 20 bp, Artificial Sequence
  irp2 deletion construct
atgacggagtcaccgcaaac

SEQ ID NO: 24 - DNA, 20 bp, Artificial Sequence
  irp2 deletion construct
cggtatagccgacctttctg

SEQ ID NO: 25 - DNA, 20 bp, Artificial Sequence
  irp2 deletion construct
ttacatcacgcgtttaaagg

SEQ ID NO: 26 - DNA, 20 bp, Artificial Sequence
  irp2 deletion construct
atggataacttgcgcttctc

SEQ ID NO: 27 - DNA, 20 bp, Artificial Sequence
  iucABCDiutA deletion construct
gagccgccccaaacgacagc

SEQ ID NO: 28 - DNA, 20 bp, Artificial Sequence
  iucABCDiutA deletion construct
ggcttttctgataccaatct

SEQ ID NO: 29 - DNA, 19 bp, Artificial Sequence
  iucABCDiutA deletion construct
aagcacaatcaatatatgg

SEQ ID NO: 30 - DNA, 20 bp, Artificial Sequence
  iucABCDiutA deletion construct
ccggtattcctttacaacaa

SEQ ID NO: 31 - DNA, 20 bp, Artificial Sequence
  iroA(iroNDCB) deletion construct
tcctgttggccagcgtctat

SEQ ID NO: 32 - DNA, 20 bp, Artificial Sequence
  iroA(iroNDCB) deletion construct
ctccttcagcccgaacaaac

SEQ ID NO: 33 - DNA, 20 bp, Artificial Sequence
  iroA(iroNDCB) deletion construct
tcgtaattattaggactaag

SEQ ID NO: 34 - DNA, 20 bp, Artificial Sequence
  iroA(iroNDCB) deletion construct
gctcctgtatactatggcag

SEQ ID NO: 35 - DNA, 20 bp, Artificial Sequence
  iroA(iroBCDN) deletion construct
tcagaatgatgcggtgacac

SEQ ID NO: 36 - DNA, 18 bp, Artificial Sequence
  iroA(iroBCDN) deletion construct
tcaaccttttagtaaacc

SEQ ID NO: 37 - DNA, 20 bp, Artificial Sequence
  iroA(iroBCDN) deletion construct
gtccggcggtaacttcagcc

SEQ ID NO: 38 - DNA, 19 bp, Artificial Sequence
  iroA(iroBCDN) deletion construct
atgctgaacatgcaacaac

SEQ ID NO: 39 - DNA, 20 bp, Artificial Sequence
  kfuABC deletion construct
gcagcagatgaatattctgg

SEQ ID NO: 40 - DNA, 20 bp, Artificial Sequence
  kfuABC deletion construct
ttccgacgccaatgctgatc

SEQ ID NO: 41 - DNA, 20 bp, Artificial Sequence
  kfuABC deletion construct
tctgggtgcagaaccaaatg

SEQ ID NO: 42 - DNA, 20 bp, Artificial Sequence
  kfuABC deletion construct
atggagtggtagtacgttgg

SEQ ID NO: 43 - DNA, 20 bp, Artificial Sequence
  tonB deletion construct
agcaacttaacgctggcagc

SEQ ID NO: 44 - DNA, 19 bp, Artificial Sequence
  tonB deletion construct
tgagctgggtaccaacacc

SEQ ID NO: 45 - DNA, 19 bp, Artificial Sequence
  tonB deletion construct
gcaatcatattcaataagg

SEQ ID NO: 46 - DNA, 20 bp, Artificial Sequence
  tonB deletion construct
taaaacgctgcggcggaccg

SEQ ID NO: 47 - DNA, 20 bp, Artificial Sequence
  salmochelin receptor IroN from iroA(iroNDCB)
gaaaatccctcttttaacgc

SEQ ID NO: 48 - DNA, 20 bp, Artificial Sequence
  salmochelin receptor IroN from iroA(iroNDCB)
tcagaatgaaactaccgccc

SEQ ID NO: 49 - DNA, 20 bp, Artificial Sequence
  salmochelin receptor IroN from iroA(iroBCDN)
tcgatccggttgtttgcagg

SEQ ID NO: 50 - DNA, 20 bp, Artificial Sequence
  salmochelin receptor IroN from iroA(iroBCDN)
tcagaatgatgcggtgacac

SEQ ID NO: 51 - DNA, 20 bp, Artificial Sequence
  irp2 complementation
ccccttcgacctttaaacgc

SEQ ID NO: 52 - DNA, 20 bp, Artificial Sequence
  irp2 complementation
ctatatccgccgctgacgac

SEQ ID NO: 53 - DNA, 20 bp, Artificial Sequence
  iucABCDiutA complementation
ccagtacagggatcgcgacc

SEQ ID NO: 54 - DNA, 24 bp, Artificial Sequence
  iucABCDiutA complementation
tcagaacagcacagagtagttcag

SEQ ID NO: 55 - DNA, 20 bp, Artificial Sequence
  tonB complementation
cgtaaagcacggcaaagctc

SEQ ID NO: 56 - DNA, 19 bp, Artificial Sequence
  tonB complementation
tcagttaatctcgacgccg